(19) United States
(12) Patent Application Publication
(10) Pub. No.: US 2024/0428928 A1
(43) Pub. Date: Dec. 26, 2024

(54) SYSTEM AND METHOD FOR ASSISTING IN PEER REVIEWING AND MANUAL CONTOURING OF MEDICAL IMAGES

(71) Applicant: Boston Scientific Scimed, Inc., Maple Grove, MN (US)

(72) Inventors: Djamal BOUKERROUI, Oxford (GB); Mark John GOODING, Oxford (GB); Marcel VAN HERK, Manchester (GB)

(21) Appl. No.: 18/699,292

(22) PCT Filed: Sep. 28, 2022
(86) PCT No.: PCT/EP2022/077021

§ 371 Date: Apr. 7, 2024

(30) Foreign Application Priority Data
Oct. 7, 2021 (GB) 2114399.5
May 30, 2022 (GB) 2207977.6

(51) Int. Cl. G16H 30/40; G06V 10/46; G06V 10/56; G06V 10/60

(52) U.S. Cl. CPC G16H 30/40; G06V 10/46; G06V 10/56; G06V 10/60

(57) ABSTRACT

A method and system for contouring an image for display is described. The method comprising: providing at least one image to be contoured; providing a window width parameter, W, and a window level parameter, L; displaying the medical image according the W,L parameters; performing the following steps: determining local image intensities of the at least one image for each position of the contour, as it is generated; determining if the parameters are suitable for each position of the contour on the image as it is generated; according to the determined local image intensity; if the parameters are not suitable, alerting the user that at least one of the parameters is not suitable for a most recently generated position on the contour; adjusting at least one of the—parameters to be suitable for the most recently generated position on the contour; and displaying the—image according to the adjusted settings.

This invention relates to the fields of medical imaging. In particular, the contouring, reviewing or editing of an anatomical or target structure(s) for radiotherapy treatment planning by, for example, a radiation oncologist on a medical image of the patient. The invention provides tools to ensure that the user is using an appropriate scaling of the display of the medical image when contouring a structure or reviewing the contour of a structure on the medical image.

### BACKGROUND

In medical imaging it is sometime necessary to delineate a structure or other object on a medical image of the patient. For example, in radiotherapy treatment planning it is necessary to take a volumetric (3D) image of the patient, on which the treatment plan can be designed, and the radiation dose delivery estimated. A 3D image/volumetric image is an image comprising a volume, normally made from multiple 2D images in medical imaging. Elements of a 3D image are known as voxels. A 2D image is an image comprising a slice in a 2D plane. This slice may have a thickness defined, and thus constitute a volume in a medical image as a cross-section of a patient. Elements of a 2D image are normally referred to as pixels.

Such a 3D image is called a simulation CT (Computed Tomography) image. Part of this process involves the outline of anatomical and target structures on the image of the patient such that the estimated radiation dose delivery to each of the structures can be calculated and reported back to the clinical staff doing the treatment planning. In radiotherapy planning this image is normally a CT image, but MRI (Magnetic Resonance Imaging) images may also be used.

The size and the location of a tumour as indicated on the image are important indicators contributing to the decision process on the choice of the best treatment delivered to the patient. An accurate delineation of both healthy organs and cancerous regions on the patient image, is required in order to make the best clinical choice. For example, in radiation treatment planning accurate delineation is required when planning maximize the delivered dose of radiation to the tumour while minimizing the dose on healthy tissues.

The outlining or delineation process is known in the field of radiation oncology as “contouring”. The wording “segmentation” may also be used, and this may be used to refer to both the process and the outcome. Auto-segmentation or auto-contouring refers to when the contouring task is performed automatically by an image processing system (Sharp, 2014, Lustberg, 2018). Semi-automatic or interactive/manual tools also exist (Ramkumar et al., 2016).

Automation in the contouring process helps to reduce delineation time but also to improve image segmentation consistency and reduce inter- and intra-observer variability (Brouwer et al. 2012, Vinod et al. 2016, 2016a, Patrick et al. 2021). However current automatic solutions still require manual editing to correct for any error of the automatic system (Lustberg et al., 2018, Brouwer et al. 2020). Furthermore, automatic solutions are still limited in terms of the anatomical regions that can be contoured or image modalities that are supported. Thus, manual contouring is still performed on daily basis in the clinic.

Manual contouring is typically performed using a workstation with an appropriate image display. Although nowadays most medical imaging techniques are three dimensional, typically comprising a stack of 2D medical images slices, contours may be drawn around each structure image slice by image slice. The 3D image is normally displayed slice by slice on a screen to one of the 3 planes, Axial, Sagittal, Coronal (these are well known anatomical planes in medical imaging) or according to the image acquisition plane. The image may also be displayed by resampling in any plane. Multiple 2D images can also be displayed at the same time and on the same screen or on multiple screens. Then the clinician may manually generate new contours, or review and/or edit an existing set of displayed contours.

Contours are generally displayed as an overlay on the medical image slice. The contour can be filled or displayed as curves with a certain line thickness. Colour is generally used to improve contrast as most of medical images are scalar valued (non-colour) images and are therefore displayed in grayscale. Medical images generated by current medical imaging techniques, including X-Ray, CT, MRI PET (Positron Emission Tomography) typically have scalar values encoded with 12 to 16 bits per value (pixel). This corresponds to 4,096 to 65,536 stored values. These stored values can then be mapped to real-world values. For example, a CT image may have a stored value of 1024, which could be mapped to a real-world unit of value of 0 Hounsfield Units. Furthermore, for some 3D image modalities different mapping to real-world values may be applied to each slice such that the dynamic range of the 3D image is apparently much larger. For example, a PET image may have a stored value of 1024 mapping to 136.79 Bq/mL (Becquerel per milliliter) on one slice of the image, while on a different slice a stored value of 1024 may map to 2440.99 Bq/mL. This conversion between stored values and real-world values is determined by the file format standard (e.g. DICOM) with which the medical image is stored. Further references to stored values may be interpreted to include the stored values after conversion to real-world units according to the medical image file format.

Thus, the range of stored values in medical images is far wider than what current medical displays can support, with displays generally supporting 8 bits to 10 bits. Guidelines and recommendations for the assessment of display quality for display devices used in medicine are regularly published and updated (Bevins et al. 2019). Practical recommendation how to implement a quality assurance process according to these guidelines are also available (Bevins et al. 2020). Interestingly most of the pattern and quality assessment processes described in (Bevins et al. 2019) use an 8-bit display (256 shades of gray); This is not surprising as a stronger limitation on visualizing the image is the inability of human observers to discriminate a wider range of simultaneous shades of gray (between 700 to 900 according to Kimpe and Tuytschaever 2007).

Because of these two limitations-display technology and human perception—common practice in imaging technologies is to define a display range, known as a window, in which the image intensities are mapped to displayed shades of grey. This is normally defined by two values:
W: A window width: this is typically the length of the dynamic range of intensities (For example, a window from a to b will have a width W=b−a).
L: A window level: the intensity value at centre of the window. (For example, a window from a to b will have L=(a+b)/2.)

The image is then scaled such that only image intensity values between L−W/2 and L+W/2 are rendered on the display. The restriction of a numerical value to a given range, here between L−W/2 and L+W/2, is known as clamping. Clamping is the restriction of a numerical value to a given range [a b]. Specifically, the value is linearly scaled if it is within the given range; it is set to the lower value, a, if it is lower than a; it is set the higher value, b, if it is greater than b. Intensity values lower than L−W/2 will be clamped and all will be displayed as the lowest value (black colour) on the screen. Similarly, any intensity value greater than L+W/2 will be also clamped and will be displayed as the highest value (white colour) on the display. Intermediate image intensity values will be scaled, normally linearly, and will cover the full range of display of the device. Such transformation is generally referred by window/leveling or a clamped linear intensity transformation. In some cases the window level parameters W,L are also referenced simply as window. As example wording such the Lung Window or the Bone Window will refer to commonly used or recommended W,L settings to display lung tissue or the bone anatomy. Non-clamped intensity values for a (W,L) setting are intensity values ranging from L−W/2 to L+W/2, generally denoted as [L−W/2, L+W/2], also referred simply by the word Window.

Note that scalar intensity values are often displayed as shade of grey, however colour mapping may be used to convert a scalar intensity value to a colour display range. Thus, the term “shades of grey” should be interpreted as scalar image values prior to mapping to a display colour.

Recommendations on what is the useful range of shades of gray given the clinical question that the medical practitioner need answering or given the task at hand. As for example, the useful range of intensity values to look at the lung structures on a CT scan is different from the one for liver structures or the head and neck structures.

Guideline for appropriate values for W, L for CT are available (Bongartz, 2000). As an example, for general CT chest exam, the recommended window width (W) for the soft tissues is in between 300 to 600 Hounsfield Units (HU) with a window level (L) between 0 to 30 HU. However, the recommended window width (W) for lung parenchyma is in between 800 to 1,600 HU with a window level (L) between −500 to −700 HU. Specific example values for displaying lung tissue could be to set W=1000 HU and L=−500 HU, thus defines a range of intensities from [−500-1000/2, −500+1000/2]=[−1000, 0], and is referred by the Lung Window.

FIG. 3, shows a few example intensity transformations for some typical window W, L parameter sets recommended for displaying a CT image. Examples of recommended (W,L) parameter sets for CT are shown in this figure for: Bone anatomy 304, Liver 305, Chest general 306 and Lung 307. For clarity of the drawing, the x axis 313 is also drawn at 314. This x-axis represents stored image values that have been mapped to real-world (Hounsfield Unit) values; the shown range (from −000 to 2000 HU) is typical of CT imaging. The y axis 311, graded in percentage, gives the corresponding shade of gray as illustrated by the black to white color bar 310. The window levels 308 of the different transformations always rendered to the middle (50%) of the shades of gray 301. The window level, L, 308 and the window width, W, 209 are shown for the bone anatomy transformation 312, drawn with a thick line. On this example, the clamped linear intensity transformation was shown for L=480 HU 208 and W=2500 HU 309, thus defining a range of non-clamped image intensities from −770 to +1700 HU to be linearly scaled to be displayed between 0% to 100% of the shades of gray. Image intensities lower than −770 HU 316, will be clamped and will be displayed as the lowest value (black colour) on the display; Image intensities higher than +1700 HU 215 will also be clamped and will be displayed as the highest value (white colour) on the display. While in the clinic, the quality of the displaying device is regularly checked, and the clinical teams are trained and educated in guidelines on the appropriated (W,L) parameters to use, in some cases the clinician may need to change the (W,L) settings several times while contouring. Indeed, an organ or a tumour may be surrounded by different tissue types that generally requires different (W,L) settings to ‘optimally’ display and visualise the boundary between normal tissue and tumour tissue on the screen. As an example, a lung tumour invading the chest wall near a rib, as illustrated in FIG. 11. Different (W,L) settings are needed to assess the correct boundaries between the different tissue types. For example window/level setting typically used for a lung may be best to assess the boundary between the tumour 1101 and the lung 1102, while a window/level setting typically used for a bone may be best to assess the boundary between the tumour 1101 and the rib 1103.

To illustrates this further, FIGS. 12, a, b, c and d show an example of an intensity profile of a CT image values, as loaded in FIG. 12-a, and after the application of a CT-Lung window 307 in FIG. 12-b, a CT-Bone window 304 in FIG. 12-c and a CT-Chest window 306, in FIG. 12-d. In this example, the intensity profile corresponds to the anatomical region shown with a dashed line on FIG. 11, starting from 1107 and ending up at 1108. The CT image intensity profile 1201 is shown on FIG. 12-a. The y-axis, 1203, represents the loaded CT image intensity values in Hounsfield Units. The x-axis 1202 represents the spatial position graded in pixels; the first pixel on the intensity profile corresponding to the position 1107, located in the lung, and the last pixel to the position 1108, located in the image background. As can be seen on FIG. 11, the line profile crosses several tissue types: lung, tumour, bone, fat, muscle, fat and finally air (outside the body). These different regions can be observed on the line profile 1201; they are labelled by the letters A to F and are summarised in the legend 1210. The boundaries between the different tissues are illustrated by the vertical dashed lines, labelled from 1204 to 1209. FIG. 12.b shows the intensity profile 1221 after application of the CT-Lung window with (W,L)=(1000, −500), 307, for display. The x-axis 1222 is the same as 1202 on FIG. 12-a. The boundaries between the different tissues, here labelled from 1224 to 1229, are also at the same positions as on FIG. 12-a. The y-axis, 1223, graded in percentage gives the corresponding shades of gray as illustrated by the black to white colour-bar 1230. The intersection of the intensity profile, 1221, with the horizontal lines 1231 and 1232 indicates clamped pixels. On this example, the boundary, 1225, between the tumour and the bone cannot be seen on the displayed image as both the tumour and bone intensity values are outside the range of displayed values and are therefore clamped. Also, the boundaries, 1227 and 1228, between the muscle tissue and the surrounding fat tissue cannot be drawn accurately because of the clamping image intensities of muscle pixels and also because of the low image contrast at the boundary locations as both tissues are displayed in the highest shades of gray. With a lung window, the image contrast is adequate to appreciate the correct boundary locations of 1224 and 1229.

FIG. 12-c shows the intensity profile, 1241, after application of the CT-Bone window with (W,L)=(2500, 480), 304. The x-axis 1242 is the same as 1202 on FIG. 12-a. The boundaries between the different tissues, here labelled from 1244 to 1249, are also at the same positions as on FIG. 12-a. The y-axis, 1243, graded in percentage gives the corresponding shades of gray as illustrated by the black to white colourbar 1250. The intersection of the intensity profile 1241 with the horizontal lines 1251 and 1252 indicates clamped pixel intensities. On this example, only the boundaries with the bone tissue, 1245 and 1246 have an adequate contrast to be drawn or reviewed accurately. The large window width of the bone window, W=2500, makes it impossible to discriminate between soft tissues (muscle, tumour, fat, etc) thus boundaries such as 1247 and 1248 cannot be drawn or reviewed accurately. An accurate discrimination between soft tissues will require to display the image with a smaller W value. This is illustrated on FIG. 12-d showing the intensity profile, 1261, after application of the CT-Chest window with (W,L)=(350, 40), 306. The x-axis 1262 is the same as 1202 on FIG. 12-a. The boundaries between the different tissues, here labelled from 1264 to 1269, are also at the same positions as on FIG. 12-a. The y-axis, 1263, graded in percentage gives the corresponding shades of gray as illustrated by the black to white colour-bar 1270. The intersection of the intensity profile 1261 with the horizontal lines 1271 and 1272 indicates clamped pixels. On this example lung tissue, A, and the image background, F, were clamped making it difficult to review or draw contours at boundary locations 1264 or 1269. Also, the bone tissue, C, is clamped making it difficult to appreciate the correct location of the bone boundaries, 1265 and 1266.

Current medical image visualisation devices allow a user to quickly switch between different (W,L) settings; There is also the possibility for the user to freely and interactively (via a computer mouse for example) adjust the contrast of the displayed image. In other words, the user is setting a custom (W,L) parameters for the displayed image. Such technical features helps but they are limited as there is nothing that prevents the user from contouring or reviewing or editing contours with non-suitable (W,L) parameters. Furthermore, guidelines for specific (W,L) parameters can only be defined on an imaging modality with standardised intensity values such as Hounsfield Units for Computer Tomography; MRI is not a quantitative technique and the range of acquired image intensities is highly variable. In other words, current solutions facilitate contouring by allowing variable (W,L) to be applied to the displayed image to improve contrast visualisation but do not actively inform the user that contouring is being performed on a sub-optimal image window width/level. Thus, this may be a cause of errors in the contouring and reviewing process. If such errors are not detected, the radiotherapy treatment plan will be affected and could potentially lead to harm to the patient.

### PROBLEM SUMMARY

Thus, there is a need for a method and a system to inform users while contouring, reviewing, or editing one or more contours on a medical image that one or more of the current image window width and level parameters as used to display the image with the one or more contours are inappropriate for the task of contouring, editing or reviewing contours. Such a system and method is described and disclosed by the present invention.

According to the invention there is provided a method for contouring a medical image that is displayed on a display device, comprising the steps of: providing at least one medical image with one or more structures to be contoured; providing a window width parameter, W, and a window level parameter for the at least one medical image, L displaying the medical image according to the W,L parameters; performing the following contouring steps: determining local image intensities of the at least one medical image for each position of the contour, as the contour is generated for a structure on the image; determining if the window width parameter, W, and the window level parameter, L, are suitable for contouring for each position of the contour on the medical image as the contour is generated; according to the determined local image intensity; if at least one of the parameters are not suitable for contouring, providing an alert that at least one of the parameters W and L is not suitable for a most recently generated position on the contour; and adjusting at least one of the window width parameter, W, and the window level parameter, L, to be suitable for contouring of the structure for the most recently generated position on the contour; and displaying the medical image according to the adjusted W,L settings.

In a further embodiment of the invention there is also provided a method for reviewing a previously contoured medical image that is displayed on a display device, comprising the steps of: providing at least one medical image and at least one contour on the medical image to be reviewed; providing a window width parameter, W, and a window level parameter, L for the medical image; displaying the medical image according to the W, L parameters and displaying the at least one contour; selecting at least one contour on the previously contoured image for review; performing the following contour review steps: determining local image intensities of the at least one medical image for at least one portion of the selected contour to be reviewed; determining that at least one of the current window width parameter W and window level parameter L are not suitable for reviewing the at least one portion of the at least one contour, according to the determined local image intensity; providing an alert that at least one of the parameters W and L are not suitable for reviewing the at least one portion of the contour; and adjusting at least one of the window width parameter, W, and the window level parameter, L, to be suitable for reviewing the at least one portion of the contour; reviewing the further section of the contour portion using the adjusted parameters; and displaying the medical image according to the adjusted W, L settings.

In an embodiment of the invention, the contouring or contour reviewing steps are repeated until either the one or more structures to be contoured have been contoured, or all of the at least one selected contour on the previously contoured image has been reviewed.

Preferably, adjusting at least one of the window width parameter, W, and the window level parameter, L, to be suitable for contouring the at least one structure or reviewing the at least one portion of the contour is performed by the user. In an alternative embodiment of the invention, adjusting at least one of the window width parameter, W, and the window level parameter, L, to be suitable for contouring the at least one structure or reviewing the at least one portion of the contour is an automatic adjustment.

In an embodiment of the invention, the method further comprising the contour review step of: detecting one or more portions of the selected contour that are suitable for reviewing using the current window width and window level parameters; and reviewing the one or more contour portions that have been detected.

Further preferably, the method also comprises the step of editing or correcting one or more contour portions after the detected contour portions have been reviewed.

Preferably, the method further comprising the step of: determining if the parameters W, L are suitable for a current task of contour generation or contour review, if one or more of the detected parameters are not suitable for the current task, providing an alert that one or more of the parameters are not suitable, and suggesting at least one new parameter that is suitable for the current task.

Preferably, the parameter suggestion for the one or more parameters is automatic. Further preferably, the parameter suggestion for the one or more parameters is applied automatically.

In an embodiment of the invention, wherein the determination of the suitably of at least one of the window width and window level parameter is done with a machine learning algorithm.

Preferably, the machine learning algorithm uses information from at least one of the local image intensities and spatial information of the image.

Preferably, the method further comprising the step of: when a contour is generated or selected for review, determining a patch around either the most recently generated part of the contour, or a part of the contour for review, and assessing the local image intensities in the patch to determine if at least one of the W, L parameters is suitable, or needs to be adjusted.

Further preferably, the local image intensities in the patch used to determine the parameter suitability are calculated from one or more of: mean intensity value; maximum intensity value; minimum intensity value; median intensity value; a configurable percentile of intensities; statistical measures between the distribution of intensity values and a predefined parametric function of the W, L values.

Preferably, the local image intensities, are compared to predefined, or user configurable threshold functions for at least one of the current W, L parameters.

Further preferably, the adjusting of at least one of the window width parameter, W, and the window level parameter L is performed by a user in response to one or more of: a pop up window with suggestions for new W, L settings; a context sensitive menu accessed via the display; a mouse hover over a region of the image or the contour; an interactive mechanism such as a mouse click, a keyboard hotkey or voice control.

Further preferably, the alert provided to the user is a visual or audio alert. Preferably, the alert is provided by one or more of the following: a message in a user interface; a change in the colour of the contour being generated or edited; a change in the style or thickness of the contour being generated or edited; a change in the colour of the region around the contour being generated or edited.

Preferably, the medical image is a CT scan, an MRI scan, or a PET scan.

Further preferably, wherein when the contour has been created or reviewed, the contours of the structures are saved or provided as an output.

In an embodiment of the invention, after editing or generation of a contour has been completed the steps are repeated so that a plurality of contours are created or reviewed on a medical image.

Preferably, at least one of the window width parameter, W, and the window level parameter, L, at the start of the contouring or reviewing process are one of the following: image modality dependent; workflow dependent; set from a previous use of the system; user configured or defined from parameters of the stored image.

In an embodiment of the invention, the medical image is a 2D medical image, a 3D medical image or a time-series of medical images.

Further preferably, a minimum value of at least one of the W, L parameters is set for contouring, editing, or contour reviewing. Preferably, a maximum value of at least one of the W, L parameters is set for contouring, editing, or contour reviewing. In a preferred embodiment of the invention, the contouring is one of manual contouring, semi-automatic contouring, or automatic contouring.

In a further embodiment of the invention, there is also provided a system for analysing a medical image comprising: a display for displaying at least one medical image to be contoured; a processor for setting a window width parameter, W, and a window level parameter, L, for the display device to the correct value for the start position of the contour for a structure on the at least one medical image to be contoured; the processor determining the local image intensities of the at least one medical image for each position of the contour, as the contour is generated; the processor determining if the window width parameter, W, and the window level parameter, L, are correct for each position of the contour on the medical image as the contour is generated; according to the determined local image intensity; if at least one of the parameters is not correct, alerting the user that at least one of the parameters W and L are not correct for a current position on the contour; and adjusting at least one of the window width parameter, W, and the window level parameter, L to be correct for the current position on the contour.

In a further embodiment of the invention, there is also provided a system to allow a user to review a previously contoured medical image comprising: a display for displaying at least one previously contoured medical image to be reviewed with a default window width parameter, W, and a window level parameter, L; a processor for selecting at least one contour on the previously contoured image for review; the processor configured to: detect one or more portions of the selected contour that are suitable for reviewing using the default window width and window level parameters; thus allowing a user to review the one or more contour portions that have been detected; detect that at least one of the default window width parameter W and window level parameter L is not suitable for a further section of contour to be reviewed; alert the user that at least one of the parameters W and L is not suitable for the further section of the contour; and adjust the at least one of the window width parameter, W, and the window level parameter, L to be correct for the further section on the contour, to allow the user to review the further section of the contour portion using the adjusted parameters.

In an embodiment of the invention, the display displays the medical image according to the adjusted W, L settings.

Further preferably, the processor is configured to: automatically detect at least one of the current window width parameter, W, and window length parameter, L are and determine if the detected parameters are suitable for the current task of contour generation or contour review.

In an embodiment of the invention there is also provided a computer program product comprising instructions, which when the program is executed by a computer cause the computer to carry out the methods described above.

### BRIEF DESCRIPTION OF THE DRAWINGS

Further details, aspects and embodiments of the invention will be described, by way of example only, with reference to the drawings. In the drawings, like reference numbers are used to identify like or functionally similar elements. Elements in the figures are illustrated for simplicity and clarity and have not necessarily been drawn to scale.

FIG. 1—illustrates a simplified block diagram of an example of a medical imaging system.

FIG. 2—shows a simplified process of loading a medical image into a visualisation station for manual contouring, reviewing, or editing of contours.

FIG. 3—shows common applied window (W,L) settings for displaying CT images.

FIG. 4—shows a schematic representation where intensity at contour location may be at the edges of the applied window W, L parameters of the displayed CT image.

FIG. 5—shows a schematic representation where intensity at contour location may be at the edges of the applied window W, L parameters of the displayed image with non-normalised intensity values.

FIG. 6—Illustrates a flow chart of an embodiment according to the invention.

FIG. 7—illustrates a flow chart of an alternative embodiment according to the invention.

FIG. 8—illustrates a flow chart of an alternative embodiment according to the invention.

FIG. 9-a—illustrates a schematic representation of a 2D image slice during a contouring operation.

FIG. 9-b—illustrates a schematic representation of a 2D image slice during a contour reviewing operation.

FIG. 10—illustrates a schematic representation of system suggested appropriate new window W, L parameters of the displayed image.

FIG. 11—shows a simplified stylized example of an axial image slice of a thorax of a patient.

FIG. 12-a illustrates an example of an intensity profile of a CT-image of a patient.

FIG. 12-b illustrates an example of an intensity profile of a CT-image of a patient after the application of W, L setting of a CT-Lung window for displaying the image.

FIG. 12-c illustrates an example of an intensity profile of a CT-image of a patient after the application of W, L setting of a CT-Bone window for displaying the image.

FIG. 12-d illustrates an example of an intensity profile of a CT-image of a patient after the application of W, L setting of a CT-Chest window for displaying the image.

### DETAILED DESCRIPTION

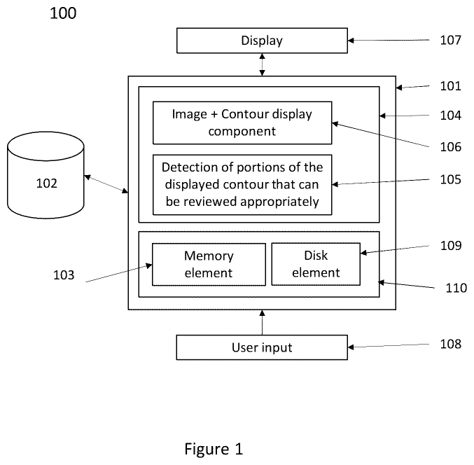

Referring now to FIG. 1, there is illustrated a simplified block diagram of an example of a medical imaging system 100 arranged to enable medical images to be displayed to a user to be used in the method of this invention. Preferably the medical images are CT images or MRI images, but other imaging methodologies such as PET scans may also be used. In the illustrated example, the medical imaging system 100 comprises one or more user terminals 101, for example comprising a workstation or the like, arranged to access medical images and/or contours stored within, for example, a database 102 or other data storage apparatus. In the illustrated example, a single database 102 is illustrated. However, it will be appreciated that the user terminal 101 may be arranged to access medical images from more than one data storage apparatus. Furthermore, in the illustrated example the database 102 is illustrated as being external to the user terminal 101. However, it will be appreciated that the user terminal 101 may equally be arranged to access medical images stored locally within a local storage module illustrated at 110 on one or more internal storage elements, such as the memory element illustrated at 103 or the disk element illustrated at 109. The user terminal 101 further comprises one or more signal processing modules, such as the signal processing module illustrated generally at 104. The signal processing module(s) is/are arranged to executing computer program code, for example stored within local storage module 110. In the illustrated example, the signal processing module(s) 105 is/are arranged to execute computer program code comprising one or more of the automated processing component(s) to detect portions of the displayed contour that are suitable for reviewing. The signal processing module 104 in the illustrated example is further arranged to execute computer program code comprising one or more image and contour display component(s) 106; the image display component(s) 106 being arranged to display the images with the window/level W, L setting and also the display of the visual alerts to the user about the suitability of the current W, L display settings for the contouring or contour reviewing process, for example on a display screen 107 or the like. The medical imaging system 100 may further comprise one or more user input devices, such as illustrated generally at 108, to enable a user to interact with computer program code etc. executing on the signal processing module(s) 104.

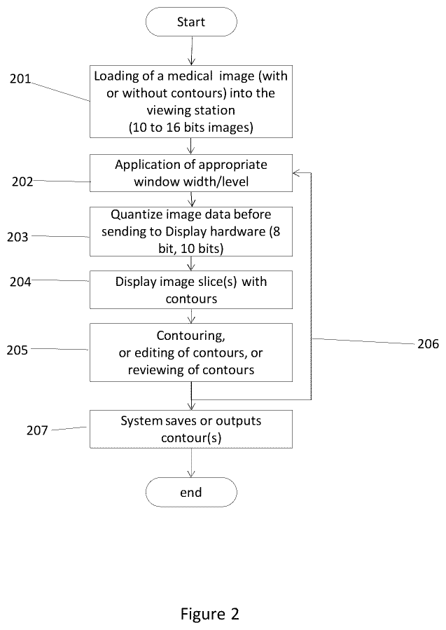

FIG. 2 illustrates a simplified process of loading a medical image into a visualisation station or other display for contouring, reviewing, or editing of contours of structures on a medical image. Preferably, this method will use the imaging system as shown in FIG. 1. The process starts with loading one or more medical images (which may be 2D or 3D, or a time-series of medical images), at step 201. It is understood that the process of loading the medical image includes the conversion between stored values to the real-world values as determined by the file format standard (e.g. DICOM) with which the medical image is stored. Preferably the medical image is one of either a CT image or an MRI image, and is a 10 to 16 bits image. Optionally, associated contours for the one or more medical images can also be loaded. These may have been for example produced by an auto-contouring system and therefore may need reviewing and potentially editing. The image intensity values are then transformed with an appropriate image window width/level 202 and are quantized (implicitly by the bit-depth of the data type used) before sending to display system (8 bits, 10 bits) 203 for displaying on a screen 204. If any existing contours are loaded with the image data, these are also displayed, preferably as an overlay on the image on the screen. The contouring, editing, or reviewing process can then start 205. The one or more contours that are generated during contouring maybe generated manually, semi-automatically or automatically. In an embodiment of the invention at least one of the image window parameters W, L may need to be adjusted one or more times while the user is contouring 206 the image or reviewing/editing an existing contour on an image. Once the contouring or reviewing process is finished, the contours are saved or stored for further use in the clinical workflow 207.

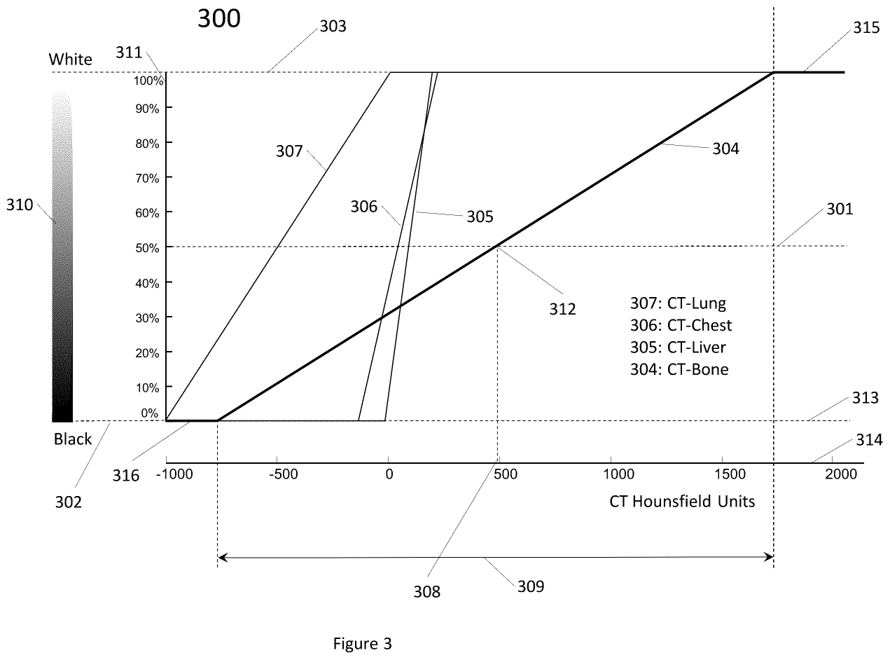

FIG. 3 shows some examples intensity transformations 300 for some typical window W, L parameter sets recommended for displaying a CT image according to an embodiment of the invention. Merely for clarity of this drawing, the x axis 313 is also drawn at 314. This axis represents the stored image values mapped to real-world values; the shown range (from −1000 to 2000 HU) is typical of CT imaging. The y axis 311, graded in percentage, gives the corresponding shades of gray as illustrated by the black to white colour-bar 310. Examples of recommended W, L parameters are shown in this figure for: Bone anatomy 304, Liver 305, Chest General 306, and Lung 307. Of course, other parameter sets may be provided for these anatomies or for other anatomical features. The window level, L, 308 and the window width, W, 309 parameters are shown for the bone anatomy intensity transformation 304. The window level, L, 308 is rendered to the middle (50%) of the available shades of gray 301 for display. For a clamped linear transformation, the window level value is always rendered to the middle (50%) of the available shades of gray. The intersection of the horizontal dashed line 301 with the linear part of the different transformations 304, 305, 306, 307 define the corresponding window level on the x axis. On this figure, the intersection is only labelled 312 for bone 304, it can be seen that there are intersections for all of the other settings, 305-307, but these are simply not labelled for ease of understanding the figure. Merely for clarity of this drawing, the clamped linear intensity transformation was highlighted for a bone window setting 304 (thick line) with L=480 HU, 308 and W=2500 HU, 309, thus defining a range of non-clamped image intensities from −770 to +1700 HU to be linearly scaled to be displayed between 0% to 100% of the shades of gray. Image intensities lower than −770 HU 316, will be clamped and will be displayed as the lowest value (black colour) on the display; Image intensities higher than +1700 HU 315 will also be clamped and will be displayed at the highest value (white colour) on the display. This clamping is not shown for other window settings, 305, 306 and 307, but should be assumed. While not commonly used for CT, the linear part of the transformation can be replaced by a non-linear transformation. Horizontal dashed lines 303 (corresponding to 100% on the y-axis) and 302 (corresponding to 0% on the y-axis) represent the maximum and minimum display values.

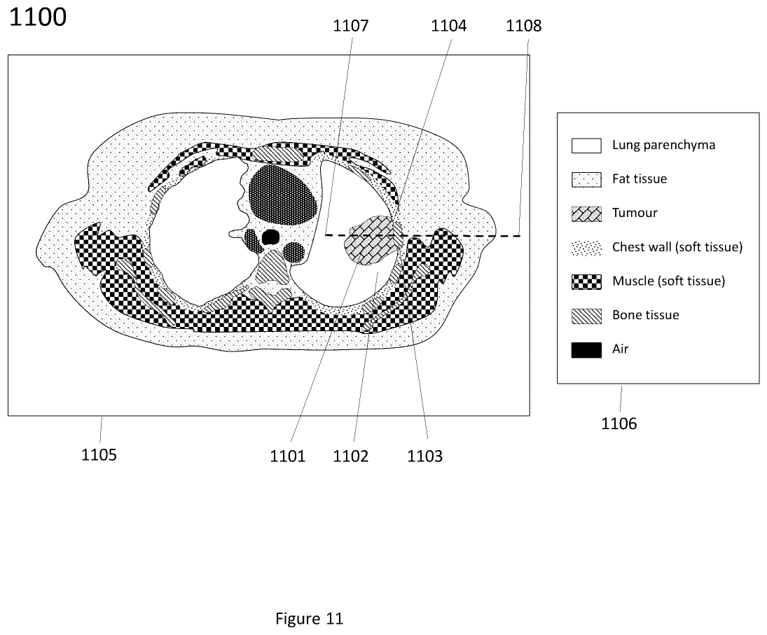

FIG. 11 shows a simplified stylized example of an axial image slice 1100 of a thorax of a patient. Several different tissue types are schematically represented in the body cross section 1105. Seven tissue types can be identified using the legend 1106. In this example, the patient has a tumour 1101 in the left lung 1102 invading the chest wall 1104 and close to a rib cage 1103. The tumour tissue abuts 3 different normal tissues types, each of the different normal tissue types will require different W, L settings to allow the boundary between the normal tissue type and the tumour tissue to be ‘optimally’ displayed on the screen. Assuming the image slice is taken from a CT scan, and as the Bone window 304 and the Lung window 307 are the two extreme window settings, a large portion of the tumour boundary will appear saturated when using either of the two windows. As for example, image intensities higher than 0 HU will be clamped when using a Lung window with (W,L)=(1000, −500); Soft tissue (including tumour tissue) and bone tissue are more likely to have an intensity values higher than 0 HU on a CT scan and will appear saturated or displayed with very low contrast on the display when using a Lung window.

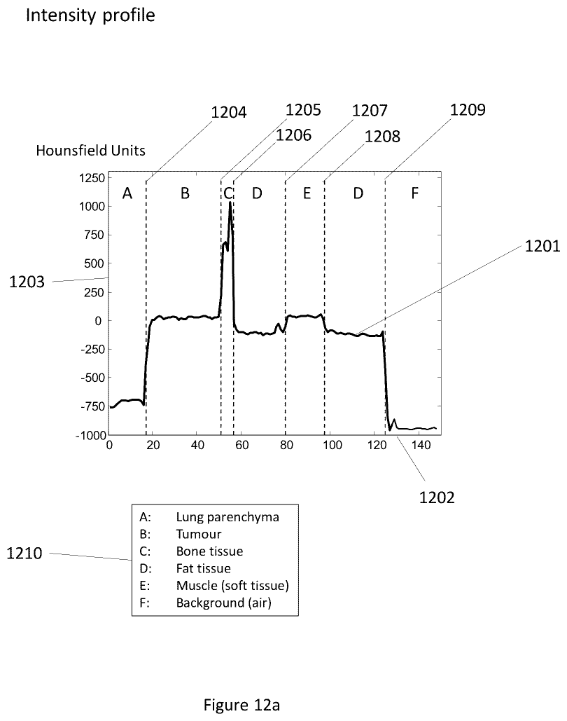

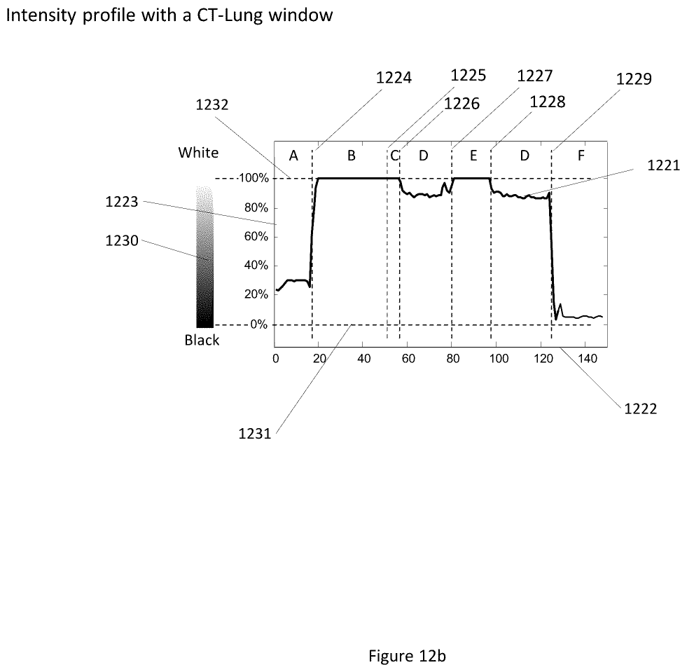

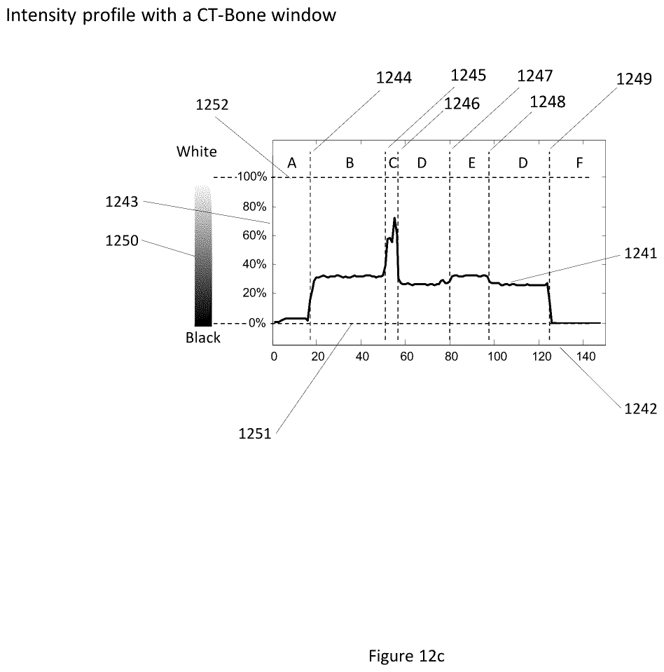

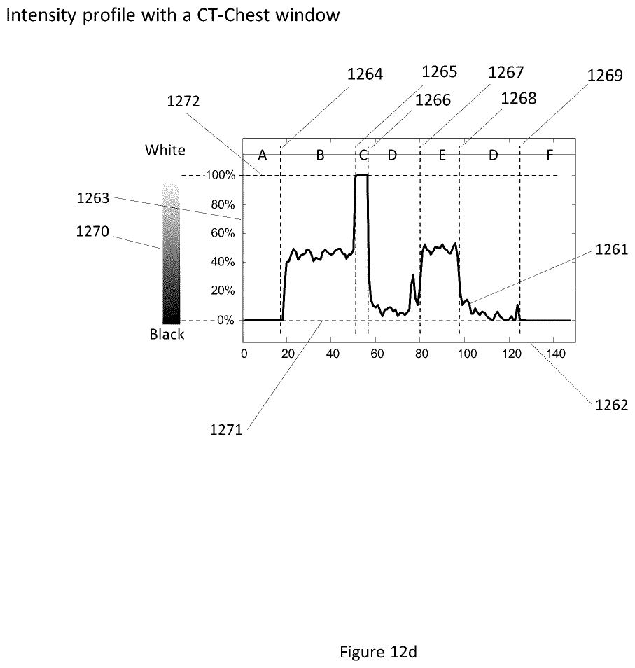

To illustrates this further, FIGS. 12, a, b, c, and d show an example of an intensity profile of a CT image values, as loaded in FIG. 12-a, and after the application of a CT-Lung window 307 in FIG. 12-b, a CT-Bone window 304 in FIG. 12-c and a CT-Chest window 306, in FIG. 12-d. In this example, the intensity profile corresponds to the anatomical region shown with a dashed line on FIG. 11, starting from 1107 and ending up at 1108. The CT image intensity profile 1201 is shown on FIG. 12-a. The y-axis, 1203, represents the loaded CT image intensity values in Hounsfield Unites. The x-axis 1202 represent the spatial position graded in pixels; the first pixel on the intensity profile corresponding to the position 1107, located in the lung, and the last pixel to the position 1108, located in the image background. As can be seen on FIG. 11, the line profile crosses several tissue types: lung, tumour, bone, fat, muscle, fat and finally air (outside the body). These different regions can be observed on the line profile 1201; they are labelled by the letters A to F and are summarised in the legend 1210. The boundaries between the different tissues are illustrated by the vertical dashed lines, labelled from 1204 to 1209. FIG. 12.b shows the intensity profile 1221 after application of the CT-Lung window with (W,L)=(1000, −500), 307, for display. The x-axis 1222 is the same as 1202 on FIG. 12-a. The boundaries between the different tissues, here labelled from 1224 to 1229, are also at the same positions as on FIG. 12-a. The y-axis, 1223, graded in percentage gives the corresponding shades of gray as illustrated by the black to white colourbar 1230. The intersection of the intensity profile, 1221, with the horizontal lines 1231 and 1232 indicates clamped pixels. On this example, the boundary, 1225, between the tumour and the bone cannot be seen on the displayed image as both the tumour and bone intensity values are outside the range of displayed values and are therefore clamped. Also, the boundaries, 1227 and 1228, between the muscle tissue and the surrounding fat tissue cannot be drawn accurately because of the clamping of muscle pixels and also because of the low image contrast at the boundary locations as both tissues are displayed in the highest shades of gray. With a lung window, the image contrast is suitable to appreciate the correct boundary locations of 1224 and 1229.

FIG. 12-c shows the intensity profile, 1241, after application of the CT-Bone window with (W,L)=(2500, 480), 304. The x-axis 1242 is the same as 1202 on FIG. 12-a. The boundaries between the different tissues, here labelled from 1244 to 1249, are also at the same positions as on FIG. 12-a. The y-axis, 1243, graded in percentage gives the corresponding shades of gray as illustrated by the black to white colourbar 1250. The intersection of the intensity profile 1241 with the horizontal lines 1251 and 1252 indicates clamped pixels. On this example, only the boundaries with the bone tissue, 1245 and 1246 have acceptable contrast to be drawn or reviewed accurately. The large window width of the bone window, W=2500, makes it impossible to discriminate between soft tissues (muscle, tumour, fat, etc) thus boundaries such as 1247 and 1248 cannot be drawn or reviewed accurately. An accurate discrimination between soft tissues will require to display the image with a smaller W value. This is illustrated on FIG. 12-d showing the intensity profile, 1261, after application of the CT-Chest window with (W,L)=(350, 40), 306. The x-axis 1262 is the same as 1202 on FIG. 12-a. The boundaries between the different tissues, here labelled from 1264 to 1269, are also at the same positions as on FIG. 12-a. The y-axis, 1263, graded in percentage gives the corresponding shades of gray as illustrated by the black to white colourbar 1270. The intersection of the intensity profile 1261 with the horizontal lines 1271 and 1272 indicates clamped pixels. On this example lung tissue, A, and the image background, F, were clamped making it difficult to review or draw contours at boundary locations 1264 or 1269. Also, the bone tissue, C, is clamped making it difficult to appreciate the correct location of the bone boundaries, 1265 and 1266.

According to an example of the invention there is provided a method for automatically detecting while contouring a structure on a medical image if the current window width/level parameters used for the displaying of the medical image provides sufficient contrast for an accurate delineation of the structure on the medical image at the correct boundary. It is anticipated that a displayed image contrast will be optimal while the user is contouring at a location where the image intensities are close to the used window level value to display the image. The image contrast will then be decreasing progressively while moving away from the window centre toward one of the two window edges where clamped image intensities will appear saturated on the screen.

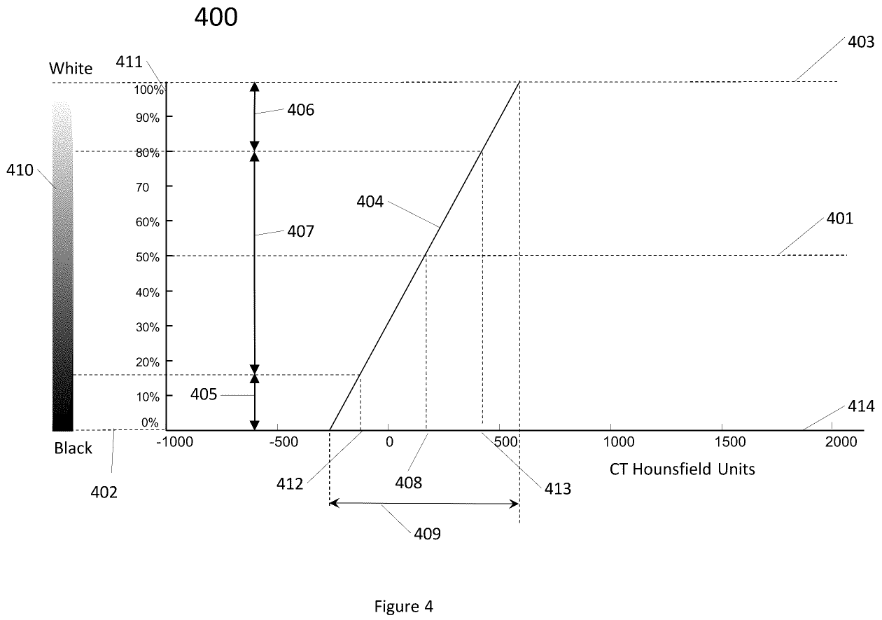

This is illustrated by the drawing on FIG. 4. This figure shows a schematic representation 400 where image intensities at contour location may be at the edges of the applied W, L parameters of the displayed image with non-normalized intensity values. The x axis 414 represents the real-world image values. The shown range (from −1000 to 2000 HU) is typical of CT imaging. The y axis 411, graded in percentage, gives the corresponding shades of gray as illustrated by the black to white colour-bar 410. The image is displayed on the display device using the W, L parameters corresponding to the intensity transformation 404 with a window width, W, 409 and a window level, L, 408; The window level L, 408 is rendered to the middle (50%) of the available shades of gray 401 for display. Image intensity values greater than the window level by half of the window width will be clamped and displayed as white 403. Image intensities lower than the window level by half of the window width will be clamped and displayed as black 402. Thus, the image contrast is assumed to be optimal only around the 50% of the range of shades of gray, represented by 407. Image intensity values lower than 412 will be displayed in the darkest shades of gray 405 and image intensity values greater than 413 will be displayed in the brightest shades of gray 406. This clamping is not explicitly shown in this figure, but can be interpreted from FIG. 3. In both ranges of shades of gray 405 and 406 the contrast of the displayed image is deemed to be inappropriate for contouring or reviewing the contour.

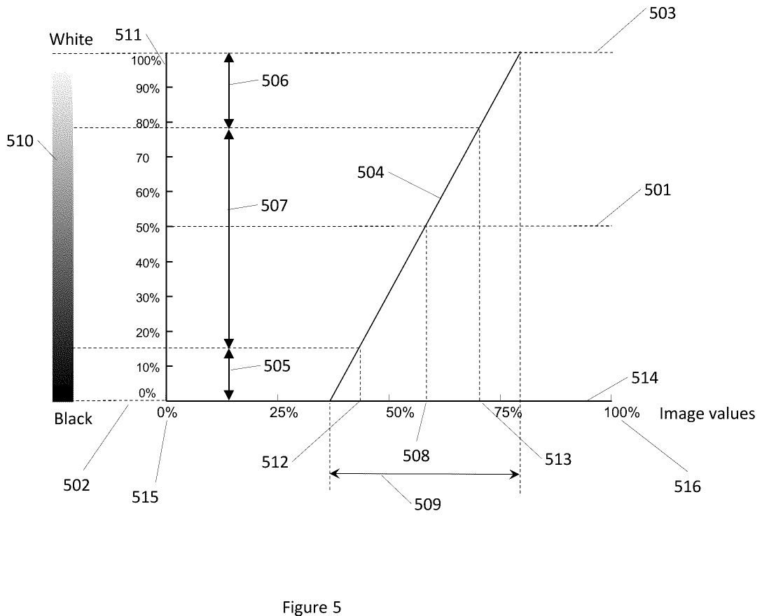

FIG. 5 is the equivalent to FIG. 4 when the imaging modality used for contouring has non-quantified intensity values, such as MRI, since image intensity values are highly dependent on acquisition and reconstruction parameters. The window width and window level are typically defined in percentage of the image intensity values. The x axis 514 represents the real-world image intensity values ranging from the minimum image intensity value to the maximum image intensity value, is graded in percentage mapping the minimum value to 0% 515 and the maximum value to 100% 516. The y axis 511, graded in percentage, gives the corresponding shades of gray as illustrated by the black to white colour-bar 510. The image is displayed using the W, L corresponding to the intensity transformation 504 with a window width, W, 509 and a window level, L, 508; The window level 508 is rendered to the middle (50%) of the available shades of gray 501 for display. Image intensity values greater than the window level by half of the window width will be clamped and displayed as white 503. Image intensities lower than the window level by half of the window width will be clamped and displayed as black 502. Thus, the image contrast is assumed to be optimal only around the 50% of the range of shades of gray, represented by 507. Image intensity values lower than 512 will be displayed in the darkest shades of gray 505 and image intensity values greater than 513 will be displayed in the brightest shades of gray 506. This clamping is not explicitly shown in this figure, but can be interpreted from FIG. 3. In both ranges of shades of gray 505 and 506 the contrast of the displayed image is deemed to be inappropriate for contouring or reviewing the contour.

The invention is a system enabling feedback to the user, while contouring, or delineating, structures on medical images, if the current window width and level used for displaying the image slices are inappropriate. Preferably the feedback is automatically provided to the user.

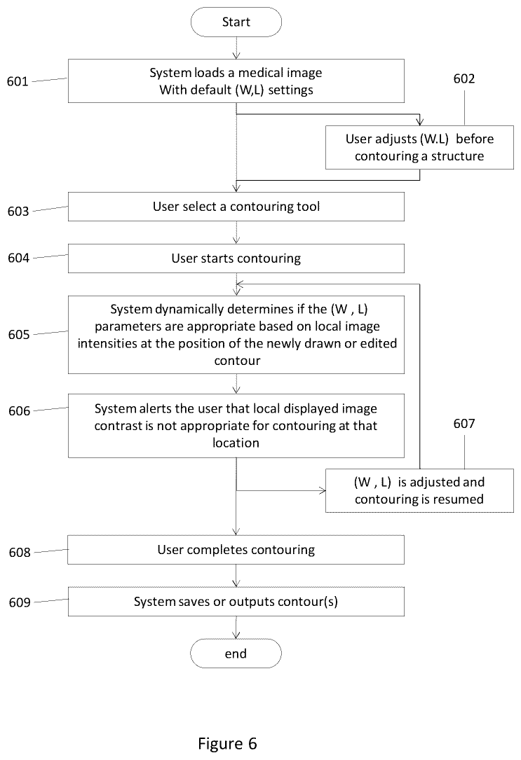

One embodiment of the invention is illustrated by FIG. 6. The system loads at least one medical image (which may be 2D or 3D, or a time-series of medical images), at step 601, the medical image will show one or more structures to be contoured. Preferably the image is a CT image or an MR image. The system applies default W, L settings for displaying the image. This image loading process may have been user initiated or may have been automatic, and the default W, L settings may be image modality dependent or workflow dependant, persistent from a previous use of the system, or user configured, or loaded from parameters of the stored image. The image is then displayed with the default W, L parameters. It is anticipated that such a system has normal viewing capabilities of the medical image visualisation tool, and the user may opt to change the view. At some point the user may want to initiate the contouring process of one or more structures on the medical image. In an embodiment of the invention the user can adjust at least one of the W, L parameters in step 602 if needed, before selecting a manual, semi-automatic or automatic contouring tool at step 603 and start contouring of the selected structure in step 604. The system and method determine local image intensities of the at least one medical image for each position of the contour, as the contour is generated. The method also determines if the current W, L parameters are appropriate, for contouring for each position of the structure on the medical image as the contour is drawn, based on local image intensities at the position of the newly drawn or edited contour in step 605, preferably this is a dynamic determination.

The system does not disturb the contouring process while the current W, L parameters are appropriate. If one or more of the W, L parameters are not suitable for contouring, the user is alerted that at least one of the parameters W and L is not suitable for a most recently generated position on the contour. The system will alert the user as soon as the newly drawn or edited contour is at an image location where locally the contrast of the at least displayed one medical image, that is at least one of the W, L parameters is not appropriate for contouring in step 606. Preferably the alert is a visual or audio alert. In an example of the invention the alert is provided by one or more of the following: a message in a user interface; a change in the colour of the contour being generated or edited; a change in the style or thickness of the contour being generated or edited; a change in the colour of the region around the contour being generated or edited.

The user may stop contouring, and adjust at least one of the W, L parameters before resuming contouring 607 of the structure on the medical image. In an example of the invention, at least one of the window width parameter, W, and the window level parameter, L are adjusted in response to the alert, to be suitable for contouring for the most recently generated position on the contour; and the medical image is displayed on the display device according to the adjusted W,L settings

In an example of the invention, the method may further comprise determining if the parameters W, L are suitable for a current task of contour generation, if the detected parameters are not suitable for the current task, an alert is provided that the parameters are not suitable, and at least one new parameter is suggested that is suitable for the current task. Preferably the alert is provided to the user.

In an example of the invention, adjusting at least one of the window width parameter, W, and the window level parameter, L, to be suitable for a current task of contour generation is performed by the user. Alternatively, adjusting at least one of the window width parameter, W, and the window level parameter, L, to be suitable for the current task is an automatic adjustment.

In an example of the invention, adjusting of at least one of the window width parameter, W, and the window level parameter L, is performed by the user in response to one or more of: a pop-up window, with the appropriate notification text and an accept/reject buttons appear on the screen; a context sensitive menu accessed via the display. The menu for parameter adjustment can be accessed on shown annotation(s) next to the portion(s) of contours that have new suggested window (W,L) settings. In an example of the invention, the context sensitive menu is shown as soon as the system detects a mouse-over (also known as a mouse hover) on the portion of contour that have a new suggested window W, L settings. In an alternative example of the invention the one or more updated parameter settings are directly applied, as soon as the system detects a mouse-over the portion of contour that have a new suggested window (W,L) settings, with an option for the user to accept these with an interactive mechanism (mouse click, a keyboard hotkey or a voice control). In a further example of the invention, the system is equipped with an audio feedback and a voice control system to apply/accept/reject the suggested W, L parameter settings. In a yet further example of the invention, a separate pane in the User Interface of the system is used to list all the new suggestions with options to navigate and apply/accept/reject the suggested setting.

In an embodiment of the invention, the user may repeats step 604-607 to complete contouring of the structure, at step 608. In an example of the invention, the process of contouring structures (steps 602-608) may be repeated for contouring multiple structures on the medical image, so that a plurality of contours are created on the medical image. The system will then save, store, or output the delineations/contours of structures created by the user for further use in the clinical workflow 609.

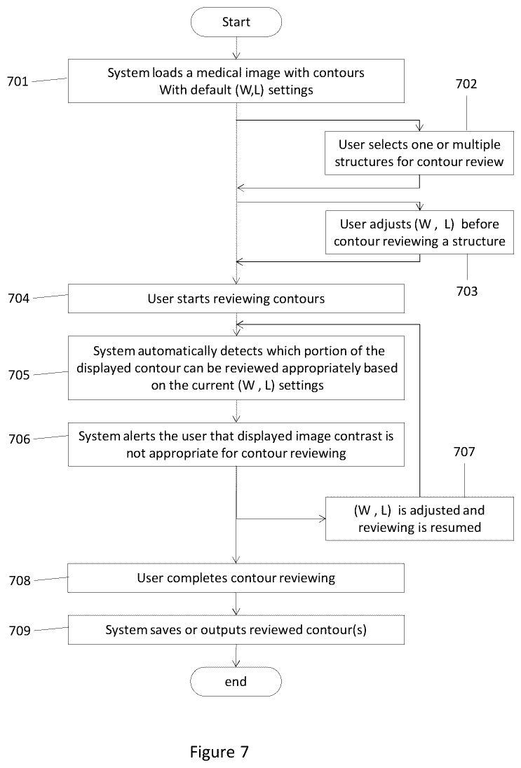

A second embodiment of the invention is illustrated by FIG. 7. This embodiment provides a system and method for the review of existing contours on a previously contoured medical image. The system loads at least one a medical image (which may be 2D or 3D, or a time-series of medical images), with the associated existing set of contours for review in step 701. Preferably the images are CT images or MRI images. The system applies default, or pre-configured W, L settings or W, L parameters stored in the image file for displaying the image with the associated contours. It is anticipated that such a system has normal viewing capabilities of the medical image visualisation tool, and the user may opt to change the view. This image loading process may have been user initiated or may have been automatic, and the default W, L settings may be image modality dependent or workflow dependant, persistent from a previous use of the system, or user configured, or loaded from parameters of the stored image. The image is then displayed with the default W, L parameters.

Optionally, the user may first select one or multiple structures for contour review 702 and then adjust at least one of the W, L parameters in step 703 if needed before starting the contour reviewing process in step 704. The system and method determine local image intensities of the at least one medical image for each position of the contour, for the contour to be reviewed. The system detects which portion(s) of the displayed contour(s) can be reviewed appropriately and which portion(s) of the contour(s) cannot be reviewed appropriately based on the current W, L settings 705. Preferably, this is detected automatically. The system does not disturb the contour reviewing process while the current W, L parameters are appropriate. When necessary, the system alerts the user that displayed image contrast is not appropriate for reviewing the displayed contours 706. If it is determined that one or more of the W, L parameters are not suitable for reviewing at least one portion of the existing contour, according to the determined image intensity, the user is alerted that at least one of the parameters W and L are not suitable for reviewing of the at least one portion of the contour. In an example of the invention, at least one of the window width parameter, W, and the window level parameter, L are then adjusted to be suitable for reviewing the at least one portion of the contour, and the reviewing further sections of the contour using the adjusted parameters.

In an example of the invention, the method may further comprise determining if the parameters W, L are suitable for a current task of contour reviewing, if the detected parameters are not suitable for the current task, an alert is provided that the parameters are not suitable, and at least one new parameter is suggested that is suitable for the current task. Preferably the alert is provided to the user.

In an example of the invention, adjusting at least one of the window width parameter, W, and the window level parameter, L, to be suitable for reviewing the at least one portion of the contour is performed by the user. Alternatively, adjusting at least one of the window width parameter, W, and the window level parameter, L, to be suitable for reviewing the at least one portion of the contour is an automatic adjustment.

Preferably the alert is a visual or audio alert. In an example of the invention the alert is provided by one or more of the following: a message in a user interface; a change in the colour of the contour being generated or edited; a change in the style or thickness of the contour being generated or edited; a change in the colour of the region around the contour being generated or edited.

In an example of the invention, adjusting of at least one of the window width parameter, W, and the window level parameter L, is performed by the user in response to one or more of: a pop-up window, with the appropriate notification text and an accept/reject buttons appear on the screen; a context sensitive menu accessed via the display. The menu can be accessed on shown annotation(s) next to the portion(s) of contours that have new suggested window (W,L) settings. In an example of the invention, the context sensitive menu is shown as soon as the system detect a mouse-over (also known as a mouse hover) the portion of contour that have a new suggested window W, L settings. In an alternative example of the invention the settings are directly applied, as soon as the system detects a mouse-over the portion of contour that have a new suggested window (W,L) settings, with an option for the user to accept these with an interactive mechanism (mouse click, a keyboard hotkey or a voice control). In a further example of the invention, the system is equipped with an audio feedback and a voice control system to apply/accept/reject the suggested setting. In a yet further example of the invention, a separate pane in the User Interface of the system is used to list all the new suggestions with options to navigate and apply/accept/reject the suggested setting.

The user may stop reviewing, and adjust at least one of the W, L parameters before resuming the contour review process (707). The user completes contour reviewing, at step 708. The contour reviewing process (steps 702-708) may be repeated for multiple structures on the medical image, so that a plurality of contours is reviewed on the at least one medical image. The system will then save, store, or output the reviewed contour(s) by the user for further use in the clinical workflow 709.

During the contour review process (steps 702-708), the user may need to adjust one or more of the displayed contours. Preferably, this contour adjustment is done manually, but it may also be done semi-automatically or automatically. The user then can switch to the contouring process described in FIG. 6. Such transition may be triggered as soon as the user selects a contouring tool. Preferably the transition is automatically triggered. The contour reviewing process can also be resumed once the user quits the contouring mode by for example deselecting the contouring tool. Preferably, the contour reviewing is automatically resumed. In an example of the invention, adjusting at least one of the window width parameter, W, and the window level parameter, L, to be suitable for reviewing the at least one portion of the contour is performed by the user. Alternatively, adjusting at least one of the window width parameter, W, and the window level parameter, L, to be suitable for reviewing the at least one portion of the contour is an automatic adjustment.

In these examples of the invention for contour generation and contour review, a minimum value of at least one of the W, L parameters is set for contouring, editing, or contour reviewing. Alternatively, a maximum value of at least one of the W, L parameters is set for contouring, editing, or contour reviewing.

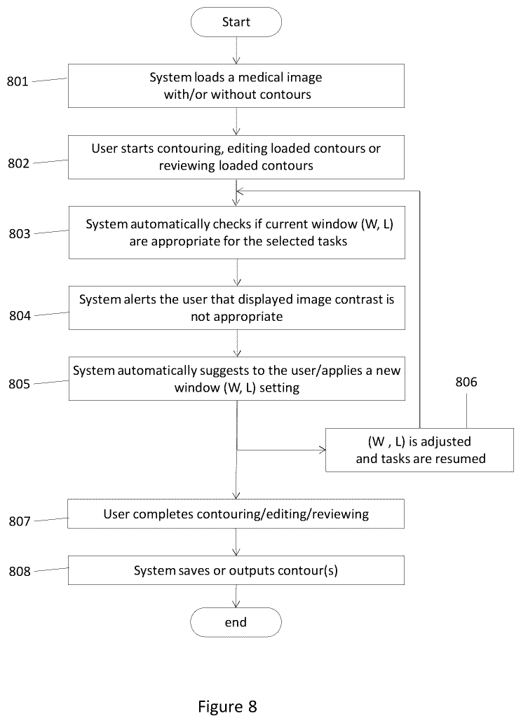

A further example of the invention is illustrated by FIG. 8. The main aspect of this embodiment is the system and method can suggest to the user at least one appropriate window W, L parameters while performing a contouring, editing or reviewing task. Preferably, this will be an automatic suggestion. The system loads at least one medical image (which may be 2D or 3D, or a time-series of medical images), with or without the associated existing set of contours in step 801. Preferably the image is a CT image or an MRI image. The user then starts one of the tasks consisting of, contouring, editing of the loaded contours or reviewing of the loaded contours in step 802. Preferably the contouring is manual contouring, semi-automatic contouring or automatic contouring. The system checks if current window W, L used to display the image and the associated contour on the screen are appropriate for the selected tasks of contouring, editing or reviewing 803 and alerts the user if the displayed image contrast is not appropriate 804. In an embodiment of the invention the alert is a visual or audio alert. In the subsequent step 805, the system suggests to the user at least one new window W, L setting that is appropriate. Preferably, when a contour is generated or selected for review, a patch around either the most recently generated part of the contour, or a part of the contour for review is determined, and the local image intensities in the patch are reviewed to determine if at least one of the W, L parameters is suitable, or needs to be adjusted.

In an example of the invention, the method may further comprise determining if the parameters W, L are suitable for a current task of contour generation, editing or contour review, if the detected parameters are not suitable for the current task, an alert is provided that the parameters are not suitable, and at least one new parameter is suggested that is suitable for the current task. Preferably, the alert is provided to the user.

It is anticipated that when the task is a contouring or editing of an existing contour, there would be no conflict on the determination of the new window parameter settings (as this will be based on the local image intensities at the single position of the newly drawn or edited contour, see step 605). Thus, the system can optionally be configured to automatically apply the at least one new parameter setting 805. For a contour reviewing task, there may be conflicts as the current window width and level may not be appropriate at different portions of the displayed contours. The system then alerts the user, that multiple settings for the W and L parameters are proposed, for him to choose appropriate settings from. Preferably the alert is a visual or audio alert. In an example of the invention the alert is provided by one or more of the following: a message in a user interface; a change in the colour of the contour being generated or edited; a change in the style or thickness of the contour being generated or edited; a change in the colour of the region around the contour being generated or edited. Optionally one or more of the settings can also be applied automatically in such scenario. The portion of interest on the contour currently being contoured or edited or reviewed can be determined automatically, for example by using eye-tracking hardware and software connected to the system to identify the location of the user's gaze or can be indicated by the position of the user's mouse on the screen. Thus, when there is a conflict between several contour locations having different appropriate W, L settings, the system can still apply the suggested appropriate setting automatically by identifying the contour of interest on the patient image, as indicated by the user's mouse or eye-tracking system. Optionally the user can adjust one or more of the W, L parameters manually and then resume the task 806.

In an example of the invention, adjusting at least one of the window width parameter, W, and the window level parameter, L, to be suitable for contouring, editing or reviewing the at least one portion of the contour is performed by the user. Alternatively, adjusting at least one of the window width parameter, W, and the window level parameter, L, to be suitable for contouring, editing or reviewing the at least one portion of the contour is an automatic adjustment.

The user completes contouring/editing/reviewing process at step 807. The steps 802-807 may be repeated for multiple structures. The system will then save, store, or output the contours for further use in the clinical workflow (808).

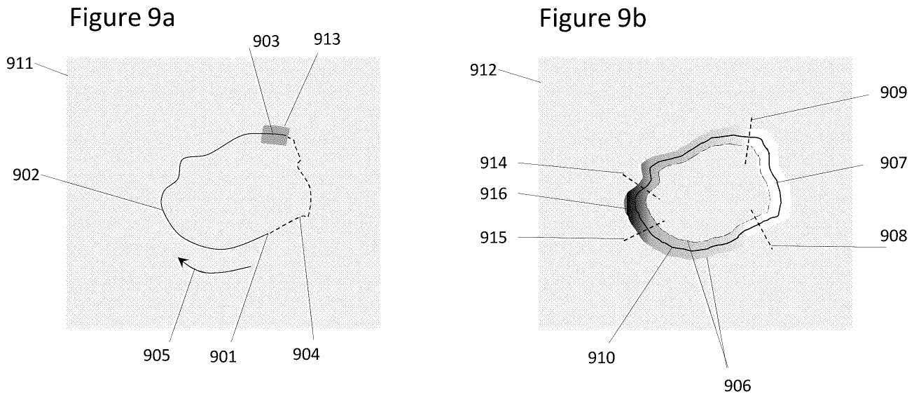

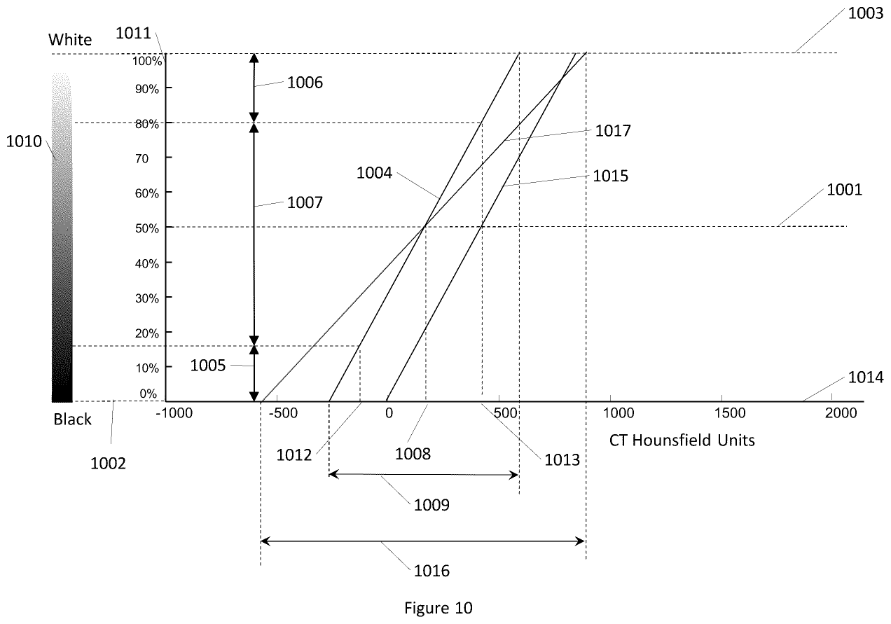

An example of the invention may also automatically detect when the window W, L parameters are not appropriate based on local image intensities. FIG. 9 and FIG. 10 (similar to FIG. 4 with new details, numbered 1015, 1016, and 1017) be used to details the underlying method. FIG. 9-a depicts a 2D image slice 911 during a manual or a semi-automatic contouring operation. The user starts contouring a structure on at image at 901 and progresses around the structure as indicated by 905. As shown the contour is generated anti-clockwise, but may be generated in other directions. The already drawn part of the contour of the structure is indicated by the continuous curve 902. The missing part (not yet drawn by the user) is indicated by the dashed curve 904. The position of the newly drawn portion of the contour is indicated by 903. The shaded part of drawing 913 is a local image patch that is preferably automatically and dynamically determined while the user is drawing the contour. The image intensities on that local patch will be analysed, preferably automatically to determine if the local image contrast is sufficient or appropriate, based on the current W, L parameter settings.

FIG. 9-b is the equivalent to FIG. 9-a but for a contour reviewing task, rather than contour generation. It shows a 2D image slice 912, with a loaded or automatically generated contour 910 that will be reviewed. The system then automatically detects that two portions of the contour 907 and 916, cannot be reviewed correctly because at least one of the current W, L settings is not appropriate for these sections of the contour. The portion 907, delimited by the dashed line segments 908 and 909, illustrates the example case where the contrast of the image at the contour location 907 is inappropriate because the current W,L parameters displays the image intensities in brightest shades of gray (as illustrated in FIG. 4 by 406 and in FIG. 5 by 506). The portion 916, delimited by the dashed line segments 914 and 915, illustrates the example case where the contrast of the image at the contour location 916 is inappropriate because the current W,L parameters displays the image intensities in darkest shades of gray (as illustrated in FIG. 4 by 405 and in FIG. 5 by 505). FIG. 9-b is illustrative as for example the system may detect that there are at least one or multiple portions of at least one or multiple contours that cannot be reviewed correctly because at least one of the current W, L settings is not appropriate. The detection relies on an automatic analysis of the local image intensities on a narrow band 906 around the loaded contour. The narrow band is split into multiple patches (overlapping or not) and then the patches are analysed, in a similarly as for the manual contouring process depicted in 900-a.

Thus, in both cases, contouring or reviewing, the detection of the W, L settings relies on the analysis of local image intensities of an image patch around the contour. As an illustrative example, suppose that the image is a CT image, the W, L parameters are as illustrated on FIG. 10, by the intensity transformation 1004, with a window width, W, 1009 and a window level, L, 1008; The window level 1008 is rendered to the middle (50%) of the available shades of gray 1001 for display. The x axis 1014 represents the real-world image intensities; The shown range (from −1000 to 2000 HU) is typical of CT imaging. The y axis 1011, graded in percentage, gives the corresponding shades of gray as illustrated by the black to white colourbar 1010. Suppose also that the minimum of the intensity values of the local patch is greater than L+W/2. Thus, all pixels in the local patch will be clamped and therefore will be saturated and displayed as white 1003. This clamping is not explicitly shown in this figure, but can be interpreted from FIG. 3. The user than will be drawing (or reviewing) a contour drawn on a pure white background, which is not appropriate to determine the boundary of any biological structure that is present in the image data as captured by the imaging sensor, but rendered incorrectly on the displaying device. Thus, the system will alert the user to this problem. Preferably the alert will be a visual or an audio alert. To improve the local contrast of such patch, where the pixels have been clamped and displayed as white, a solution is to shift the window level to adjust for this. Preferably, the parameter is shifted to higher values. This is illustrated on FIG. 10, by the intensity transformation 1015. In this example, the minimum image intensity value of the local patch is compared to L+W/2. The local contrast will be still very low if the local patch is displayed with the whitest shades of gray 1006, as only 20% the dynamic range of the shades of gray are used. This is the case for example if the minimum image intensity value of the local patch is greater than the value indicated by 1013. A similar analysis can be carried out for darkest shades of gray 1005. For example, when the maximum image intensity value of the local patch is less than the value indicated by 1012, and any pixels in the local patch with an image intensity value lower than L−W/2 will be clamped and displayed as black 1002. In this scenario an appropriate suggestion of new window setting is to shift the window level to lower intensity values.

A third scenario, is for example a large portion of pixels of the local patch (say 45%) have intensity values larger than the value (1013) and thus will be displayed with the whitest shades of gray 1006. Also a large portion of pixels of the local patch (say 45%) have intensity values lower than the value 1012 and thus will be displayed with the darkest shades of gray 1005. In this scenario, only 10% of local pixels will be displayed with shades of gray in the optimal recommended range represented by 1007. The local patch would appear as if it is binarized black and white. Thus, a good suggestion of an appropriate window setting is to keep the window level 1008 unchanged and increase the window width from 1009 to 1016 to obtain the intensity transformation 1017. The described scenario could happen if the window width was set too small to the range of available image intensities in the local patch. A fourth plausible scenario is the opposite to the third where the window width was set so large that all pixel intensities will be rendered with almost the same shades of gray. Here too the user will be drawing (or reviewing) a contour drawn on a flat gray background, which is not appropriate to determine the boundary of any biological structure that is present in the image data as captured by the imaging sensor but rendered incorrectly on the displaying device. This typical for example when drawing with a Bone window 204 the interface between soft tissues (as illustrated by the example on FIG. 12-c). Thus, a good suggestion of an appropriate window setting is certainly to reduce the window width. The window level may also need adjusting, to move it, for example, around a Central tendency estimate of the image intensities in the local patch.

For all of the different examples and embodiments of the invention, the detection that current window W, L setting are not appropriate may be made according to measurements from local image intensities of an image patch around the contour. These include at least one of:
Mean intensity value of the image patch
Maximum intensity value of the image patch
Minimum intensity value of the image patch
Median intensity value of the image patch
A configurable percentile of intensities of the image patch

Statistical measures between the distribution of image patch intensity values and a predefined parametric distribution function of W, L values.

Further preferably, the measurements from the local image intensities, are calculated before or after the application of the intensity transformation.

Further preferably, the measurements from the local image intensities, will be compared to predefined, or user configurable threshold functions of the current W, L parameters. There are many different examples of possible thresholds, for example, Minimum intensity value of the image patch>L (only the upper half of shades of gray are used) Maximum intensity value of the image patch<L. Median intensity value of the image patch>L+W/2: half of the local patch is saturated. Same for Median intensity value of the image patch<L+W/2.

Further preferably, detection that at least one of the current window W, L setting are not appropriate is done using a machine learning algorithm. The machine learning algorithm receiving the local image patch and the current window W, L parameters. Information about the local patch may include intensity measurements, as well as spatial information about the image intensities in the patch. In an example of the invention, the machine learning algorithm uses information about the local image intensity.

Further preferably a minimum allowed W value can be set when performing manual contouring, editing, or contour reviewing.

Further preferably a maximum allowed W value can be set when performing a manual contouring, editing, or contour reviewing.

Various different approaches may be implemented to alert the user to inadequate window W, L settings (step 606 of FIG. 6, step 706 of FIG. 7, step 804 of FIG. 8). In one example of the invention a warning message or symbol is displayed in the system user interface that portions of the contour are being drawn, have been drawn or are being displayed at an inappropriate window W, L setting. In a further embodiment, the colour of the contour is changed at locations where the contour is being drawn, has been drawn or displayed with an inappropriate window W, L setting. In a further embodiment, the curve style or thickness of the contour is changed at locations where the contour is being drawn, has been drawn or displayed with an inappropriate window W, L setting. In a further embodiment, colour of the region around the contour is changed at locations where the contour is being drawn, has been drawn or displayed with an inappropriate window W, L setting. In a further embodiment, symbolic annotations are overlayed on the contour is being drawn, has been drawn or displayed with an inappropriate window W, L setting. In a further embodiment, audio feedback is provided to the user that an inappropriate window W, L setting is being used. In a further embodiment, the system alerts the user when drawing by preventing the drawing of the contour. The drawing cursor may be changed to indicate that drawing cannot or should not be performed with the current window W, L setting. When reviewing contours, the system may alert the user to inadequate window W, L setting by not displaying, or changing the curve style, or thickness of the curve of portions of the contour for which the window W, L setting would be inappropriate. One or more of these approaches may be taken alone or in combination.

Various approaches may be implemented to alert the user that one or multiple window settings are proposed, for the user to choose from. In an embodiment of the invention the alert is a visual or audio alert. In one embodiment a pop-up window, with the appropriate notification text and an accept/reject buttons appear on the screen. In a further embodiment, a context sensitive menu can be accessed on shown annotation(s) next to the portion(s) of contours that have new suggested window (W,L) settings. In a further embodiment, the context sensitive menu is shown as soon as the system detect a mouse-over (also known as a mouse hover) the portion of contour that have a new suggested window W, L settings. In an alternative embodiment, the settings are directly applied, as soon as the system detects a mouse-over the portion of contour that have a new suggested window (W,L) settings, with an option for the user to accept these with an interactive mechanism (mouse click, a keyboard hotkey or a voice control). In a further embodiment, the system is equipped with an audio feedback and a voice control system to apply/accept/reject the suggested setting. In an alternative embodiment, a separate pane in the User Interface of the system is used to list all the new suggestions with options to navigate and apply/accept/reject the suggested setting. One or more of these approaches may be taken alone or in combination.

### REFERENCES

Bevins N, Flynn M, Silosky M, Marsh R, Walz-Flannigan A, Badano A. AAPM Report 270: Display Quality Assurance. American Association of Physicists in Medicine; 2019. https://www.aapm.org/pubs/reports/RPT_270.pdf
Bongartz G at al; “European guidelines on quality criteria for computed tomography”, 2000, EUR 16262 EN, https://op.europa.eu/en/publication-detail/-/publication/d229c9e1-a967-49de-b169-59ee68605f1a
Bevins N B, Silosky M S, Badano A, Marsh R M, Flynn M J, and Walz-Flannigan Al. Practical application of AAPM Report 270 in display quality assurance: A report of Task Group 270. Med Phys 2020.
Lustberg T, et al. “Clinical evaluation of atlas and deep learning based automatic contouring for lung cancer”, Radiotherapy and Oncology, 126(2):312-7, 2018.
Sharp G, et al. “Vision 20/20: perspectives on automated image segmentation for radiotherapy.” Medical physics, 41, 5, 2014.
Ramkumar, A. et al. “User Interaction in Semi-Automatic Segmentation of Organs at Risk: a Case Study in Radiotherapy.” Journal of Digital Imaging, vol. 29, pp. 264-277, 2016
Brouwer C L, Steenbakkers R J, van den Heuvel E, Duppen J C, Navran A, Bijl H P, Chouvalova O, Burlage F R, Meertens H, Langendijk J A, van't Veld A A. 3D variation in delineation of head and neck organs at risk. Radiation Oncology. 2012 December; 7(1):1-0.
Brouwer C L, Boukerroui D, Oliveira J, et al (2020) Assessment of manual adjustment performed in clinical practice following deep learning contouring for head and neck organs at risk in radiotherapy. Phys Imaging Radiat Oncol 16:54-60. https://doi.org/10.1016/j.phro.2020.10.001
Chrzan, Robert, and Andrzej Urbanik. “The assessment of diagnostic medical images using 10-bit grayscale—fact or myth?.” Polish journal of radiology vol. 83 e127-e132. 5 Apr. 2018, doi: 10.5114/pjr.2018.75877
Kimpe, Tom, and Tom Tuytschaever. “Increasing the number of gray shades in medical display systems—how much is enough?.” Journal of digital imaging vol. 20,4 (2007): 422-32. doi: 10.1007/s10278-006-1052-3
Vinod, Shalini & Min, Myo & Jameson, Michael & Holloway, Lois. (2016). A review of interventions to reduce inter-observer variability in volume delineation in radiation oncology. Journal of Medical Imaging and Radiation Oncology. 60. 10.1111/1754-9485.12462.
(a) Vinod, Shalini K. and Jameson, Michael G. and Min, Myo and Holloway, Lois C. “Uncertainties in volume delineation in radiation oncology: A systematic review and recommendations for future studies”, Radiotherapy and Oncology, Volume 121, Issue 2, 2016, Pages 169-179, ISSN 0167-8140, https://doi.org/10.1016/j.radonc.2016.09.009.
Qureshi, Bilal Mazhar et al. “Impact of Peer Review in the Radiation Treatment Planning Process: Experience of a Tertiary Care University Hospital in Pakistan” Journal of Global Oncology 2019:5, 1-7
Patrick, H. M. and Souhami, L. and Kildea, J. (2021) “Reduction of inter-observer contouring variability in daily clinical practice through a retrospective, evidence-based intervention”, Acta Oncologica, 60:2, 229-236, DOI: 10.1080/0284186X.2020.1825801

Examples of this invention may be applied to any or all of the following: Picture archiving and communication systems (PACS); Advanced visualisation workstations; Imaging Acquisition Workstations; Web-based or cloud-based medical information and image systems; Radiotherapy Treatment planning system (TPS); Radiotherapy linear accelerator consoles; Radiotherapy proton beam console.

The present invention has been described with reference to the accompanying drawings. However, it will be appreciated that the present invention is not limited to the specific examples herein described and as illustrated in the accompanying drawings. Furthermore, because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

The invention may be implemented in a computer program for running on a computer system, at least including code portions for performing steps of a method according to the invention when run on a programmable apparatus, such as a computer system or enabling a programmable apparatus to perform functions of a device or system according to the invention.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system. Therefore, some examples describe a non-transitory computer program product having executable program code stored therein for automated contouring of cone-beam CT images.

The computer program may be stored internally on a tangible and non-transitory computer readable storage medium or transmitted to the computer system via a computer readable transmission medium. All or some of the computer program may be provided on computer readable media permanently, removably or remotely coupled to an information processing system. The tangible and non-transitory computer readable media may include, for example and without limitation, any number of the following: magnetic storage media including disk and tape storage media; optical storage media such as compact disk media (e.g., CD ROM, CD R, etc.) and digital video disk storage media; non-volatile memory storage media including semiconductor-based memory units such as FLASH memory, EEPROM, EPROM, ROM; ferromagnetic digital memories; MRAM; volatile storage media including registers, buffers or caches, main memory, RAM, etc.

A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system.

The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the scope of the invention as set forth in the appended claims and that the claims are not limited to the specific examples described above.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures can be implemented which achieve the same functionality.

Any arrangement of components to achieve the same functionality is effectively ‘associated’ such that the desired functionality is achieved. Hence, any two components herein combined to achieve a particular functionality can be seen as ‘associated with’ each other such that the desired functionality is achieved, irrespective of architectures or intermediary components. Likewise, any two components so associated can also be viewed as being ‘operably connected,’ or ‘operably coupled,’ to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms ‘a’ or ‘an,’ as used herein, are defined as one or more than one. Also, the use of introductory phrases such as ‘at least one’ and ‘one or more’ in the claims should not be construed to imply that the introduction of another claim element by the indefinite articles ‘a’ or ‘an’ limits any particular claim containing such introduced claim element to inventions containing only one such element, even when the same claim includes the introductory phrases ‘one or more’ or ‘at least one’ and indefinite articles such as ‘a’ or ‘an.’ The same holds true for the use of definite articles. Unless stated otherwise, terms such as ‘first’ and ‘second’ are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

1-31. (canceled)

32. A method for contouring a medical image that is displayed on a display device, comprising the steps of:
providing at least one medical image with one or more structures to be contoured;
providing a window width parameter, W, and a window level parameter, L for the at least one medical image;
displaying the medical image according to the W,L parameters;
performing the following contouring steps:
determining local image intensities of the at least one medical image for each position of the contour, as the contour is generated for a structure on the image;
determining if the window width parameter, W, and the window level parameter, L, are suitable for contouring for each position of the contour on the medical image as the contour is generated; according to the determined local image intensity;
if at least one of the parameters is not suitable for contouring, providing an alert that at least one of the parameters W and L is not suitable for a most recently generated position on the contour; and
adjusting at least one of the window width parameter, W, and the window level parameter, L to be suitable for contouring of the structure for the most recently generated position on the contour; and displaying the medical image according to the adjusted W,L settings.

33. A method for reviewing a previously contoured medical image that is displayed on a display device, comprising the steps of:
providing at least one medical image and at least one contour on the medical image to be reviewed;
providing a window width parameter, W, and a window level parameter, L for the at least one medical image;
displaying the medical image according to the W, L parameters and displaying the at least one contour;
selecting at least one contour on the previously contoured image for review;
performing the following contour review steps:
determining local image intensities of the at least one medical image for at least one portion of the selected contour to be reviewed;
determining that at least one of the current window width parameter W and window level parameter L are not suitable for reviewing the at least one portion of the at least one contour, according to the determined local image intensity;
providing an alert that at least one of the parameters W and L are not suitable for reviewing the at least one portion of the contour; and adjusting at least one of the window width parameter, W, and the window level parameter, L, to be suitable for reviewing the at least one portion of the contour; reviewing the further section of the contour portion using the adjusted parameters; and displaying the medical image according to the adjusted W,L settings.

34. A method as in claim 32, where the contouring steps are repeated until the one or more structures to be contoured have been contoured.

35. A method as in claim 32, wherein adjusting at least one of the window width parameter, W, and the window level parameter, L, to be suitable for contouring the at least one structure or reviewing the at least one portion of the contour is performed by the user.

36. A method as in claim 32, wherein adjusting at least one of the window width parameter, W, and the window level parameter, L, to be suitable for contouring the at least one structure or reviewing the at least one portion of the contour is an automatic adjustment.

37. A method as in claim 33, further comprising the contour review step of:
detecting one or more portions of the selected contour that are suitable for reviewing using the current window width and window level parameters; and reviewing the one or more contour portions that have been detected.

38. A method as in claim 33, further comprising the step of editing or correcting one or more contour portions after the detected contour portions have been reviewed.

39. A method as in any of claim 32, further comprising the step of:
determining if the parameters W, L are suitable for a current task of contour generation or contour review,
if one or more of the detected parameters are not suitable for the current task, providing an alert that one or more of the parameters are not suitable, and suggesting at least one new parameter that is suitable for the current task.

40. A method as claimed in claim 39, wherein the parameter suggestion for the one or more parameters is automatic.

41. A method as claimed in claim 39, wherein the parameter suggestion for the one or more parameters is applied automatically.

42. A method as in claim 32, wherein the determination of the suitably of at least one of the window width, W, and window level, L, parameter is done with a machine learning algorithm; wherein the machine learning algorithm uses information from at least one of the local image intensities and spatial information of the image.

43. A method as claimed in claim 32, further comprising the step of:
when a contour is generated or selected for review, determining a patch around either the most recently generated part of the contour, or a part of the contour for review, and assessing the local image intensities in the patch to determine if at least one of the W, L parameters is suitable, or needs to be adjusted.

44. A method as claimed in claim 43, wherein the local image intensities in the patch used to determine the parameter suitability are calculated from one or more of:
mean intensity value;
maximum intensity value;
minimum intensity value;
median intensity value;
a configurable percentile of intensities;
statistical measures between the distribution of image patch intensity values and a predefined parametric function of the W, L values.

45. A method as claim 32, wherein the local image intensities, are compared to predefined, or user configurable threshold functions for at least one of the current W, L parameters.

46. A method as in claim 32, wherein the adjusting of at least one of the window width parameter, W, and the window level parameter L is performed by a user in response to one or more of:
a pop-up window with suggestions for new W, L settings;
a context sensitive menu accessed via the display;
a mouse hover over a region of the image or the contour;
an interactive mechanism such as a mouse click, a keyboard hotkey or voice control.

47. A method as in claim 32, wherein the alert provided to the user is a visual or audio alert; wherein the alert is provided by one of more of the following: a message in a user interface, a change in the colour of the contour being generated or edited; a change in the style or thickness of the contour being generated or edited; a change in the colour of the region around the contour being generated.

48. A method as in claim 32, wherein after editing or generation of a contour has been completed the steps are repeated so that a plurality of contours are created or reviewed on a medical image.

49. A method as in claim 32, wherein at least one of the window width parameter, W, and the window level parameter, L, at the start of the contouring or reviewing process are one of the following:
image modality dependent;
workflow dependent;
set from a previous use of the system;
user configured or defined from parameters of the stored image.

50. A method as in claim 1 wherein a minimum value and a maximum value of at least one of the W, L parameters is set for contouring, editing, or contour reviewing.

51. A system for analysing a medical image comprising:
a display for displaying at least one medical image to be contoured;
a processor for setting a window width parameter, W, and a window level parameter, L, for the display device to the correct value for the start position of the contour for a structure on the at least one medical image to be contoured;
the processor determining the local image intensities of the at least one medical image for each position of the contour, as the contour is generated;
the processor determining if the window width parameter, W, and the window level parameter, L, are correct for each position of the contour on the medical image as the contour is generated; according to the determined local image intensity; if at least one of the parameters is not correct, alerting the user that at least one of the parameters W and L is not correct for a current position on the contour; and adjusting at least one of the window width parameter, W, and the window level parameter, L to be correct for the current position on the contour.
